(19) United States
(12) Patent Application Publication
(10) Pub. No.: US 2013/0116553 A1
(43) Pub. Date: May 9, 2013

(54) BIOLOGICAL MEASURING APPARATUS AND BIOLOGICAL MEASURING METHOD

(71) Applicant: CANON KABUSHIKI KAISHA, Tokyo (JP)

(72) Inventors: Yuichiro Hori, Kawasaki-shi (JP); Mamoru Uchida, Yokohama-shi (JP)

(73) Assignee: CANON KABUSHIKI KAISHA, Tokyo (JP)

(21) Appl. No.: 13/668,087

(22) Filed: Nov. 2, 2012

(30) Foreign Application Priority Data
Nov. 9, 2011 (JP) 2011-245106

(51) Int. Cl. A61B 6/02

(52) U.S. Cl. 600/425

(57) ABSTRACT

A biological measuring apparatus measures physical parameters in a part of a living body by diffuse optical tomography. The apparatus includes at least one first light source and one first detector both disposed inside the living body, at least one second light source and one second detector both disposed outside the living body. A processor processes measured values and reconstructs the distribution of scattering coefficients or absorption coefficients of light interacting with the living body. The measured values are acquired from at least one measurement light beam emitted from the first light source and detected by one of the first and second detectors, and at least one measurement light beam emitted from the second light source and detected by one of the first and second detectors.

### BACKGROUND OF THE INVENTION

1. Field of the Invention

The present invention relates to biological measuring apparatuses and biological measuring methods.

2. Description of the Related Art

Non-invasive biological measurement technologies using light have been studied in the past namely in academic environments. With the advance of light source technologies, biological measurement apparatus using light have become more accessible to real-life applications. In particular, a technology, such as an X-ray CT apparatus, for photographing a cross-sectional image of a human body, for example, by using light is a critical examination tool in real clinical practices. Tissue in a living body is considered a strong scattering absorber; and light having a wavelength in an ultraviolet region to an infrared region is influenced by it and attenuates quickly. X-ray radiation on the other hand is not influenced by the absorption and scattering and may thus pass through a living body. The detection of the light allows relatively easy measurement of physical property value distribution in a living body. Accordingly, this technology has been the mainstream of the biological light measurement technologies. However, in recent years, near-infrared light beams having a wavelength region of 800 to 1000 nm have been used in Optical Coherence Tomography (OCT) because light beams in the wavelength region of 800 to 1000 nm are absorbed less by a living body (biological window). However, because of high susceptibility to scattering, the application of the OCT technology is limited to the measurement of a surface area of about 2 mm deep or less from a biological surface, such as the retina or skin of a human body.

On the other hand, in recent years, a measurement technology called a Diffuse Optical Tomography (DOT) has been advanced. The DOT technology measures light having been influenced by the scattering, which is a problem in OCT, and been passed through a living body to measure the physical property value distribution of the subject area. In this case, the physical property value (absorption coefficient, scattering coefficient) distribution of the area through which the light has passed is estimated. The optical response to the estimated distribution is simulated on the basis of the radiative transport equation of the light and is compared with an experiment value. The radiative transport equation governs light propagation in random media such as biological tissue. Hence, solutions of the transport equation can yield insights into the interaction between light and tissue. In practice, the estimated distribution from which an experimental value may be reproduced is the physical property value distribution to be solved (inverse problem). Japanese Patent application Laid-Open No. 6-221913 discloses an example of a biological measurement apparatus and method using DOT. U.S. Pat. No. 6,240,312 discloses a micro-scale imaging device that can be introduced into a human body for in-vivo imaging.

However, the measurement of physical property information in a deep part of a living body is difficult in the conventional measurement using DOT, which disadvantageous exhibits a low spatial resolution. The low spatial resolution is caused by a measurement light beam intensity reduced by extension of measurement light beam due to scattering by a living body and the substantial lack of amount of information caused by a low proportion of light passing through the area to be measured.

### SUMMARY OF THE INVENTION

According to an aspect of the present invention, there is provided a biological measuring apparatus which measures physical property information on a part of a living body by diffuse optical tomography, the apparatus includes at least one first light source and one first detector both disposed inside the living body, at least one second light source and one second detector both disposed outside the living body, and a processor which processes measured values and reconstructs distribution of scattering coefficients or absorption coefficients of light in the living body. The measured values are acquired from at least one measurement light beam emitted from the first light source and detected by one of the first and second detectors, and at least one measurement light beam emitted from the second light source and detected by one of the first and second detectors.

According to another aspect of the present invention, there is provided a method of measuring physical property information on a part of a living body by diffuse optical tomography, the method includes processing measured values acquired from at least one measurement light beam of at least one of a first measurement light beam emitted from a light source inside the living body, advances within the living body, and is detected by a detector outside the living body and a second measurement light beam detected by a detector inside the living body, and at least one measurement light beam of at least one of a third measurement light beam emitted from a light source outside the living body, advances within the living body, and is detected by a detector inside the living body and a fourth measurement light beam detected by a detector outside the living body and reconstructs the distribution of scattering coefficients or absorption coefficients of light in the living body.

According to embodiments of the present invention, a biological measuring apparatus and biological measuring method allow measurement of physical property information on a deep part of a living body by diffuse optical tomography with a higher resolution than a conventional DOT measuring apparatus.

Further features of the present invention will become apparent from the following description of exemplary embodiments with reference to the attached drawings.

### BRIEF DESCRIPTION OF THE DRAWINGS

FIG. 1 is a conceptual diagram of a configuration example of a measuring unit in a biological measuring apparatus according to a first embodiment of the present invention.

FIG. 2 is a conceptual diagram illustrating an effect of the spread of light in diffuse optical tomography according to the first embodiment of the present invention.

FIG. 3 illustrates a configuration example of the biological measuring apparatus according to the first embodiment of the present invention.

FIGS. 4A and 4B illustrate steps of reconstruction processing for acquisition of physical property value according to the first embodiment of the present invention.

FIG. 5 is a schematic diagram illustrating an internal measuring apparatus according to the first embodiment of the present invention.

FIG. 6 is a schematic diagram illustrating an external measuring apparatus according to the first embodiment of the present invention.

FIGS. 7A to 7C illustrate steps of reconstruction processing for acquisition of physical property value according to a second embodiment of the present invention.

FIG. 8 is a schematic diagram illustrating an internal measuring apparatus according to the second embodiment of the present invention.

FIG. 9 is a schematic diagram illustrating an external measuring apparatus according to the second embodiment of the present invention.

FIG. 10 illustrates measuring steps according to a third embodiment of the present invention.

### DESCRIPTION OF THE EMBODIMENTS

The present invention is based on a finding that the addition and coordination of a measurement with light irradiated from the outside of a living body and a measure with light irradiated from the inside of the living body allows a measurement of a tomography image of a living body with a higher resolution than conventional DOT measuring apparatuses. A biological measuring apparatus and biological measuring method according to embodiments of the present invention will be described below which measures physical property information on a deep part of a living body with diffuse optical tomography.

### First Embodiment

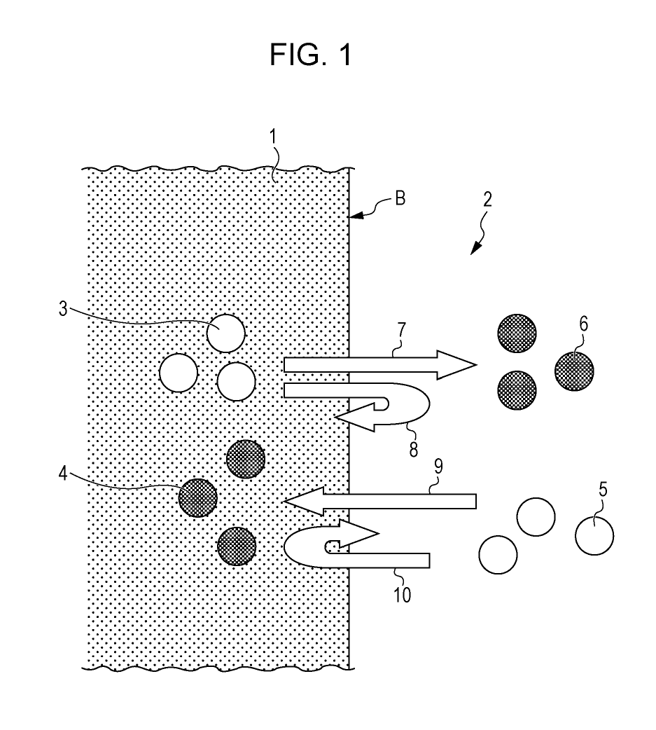

According to a first exemplary embodiment of the present invention, a configuration of a biological measuring apparatus and biological measuring method are described. First, a configuration of a measuring unit in a biological measuring apparatus according to this embodiment will be described with reference to FIG. 1. Referring to FIG. 1, the inside 1 of a living body is neighboring to the outside 2 of the living body across a boundary B. The inside 1 of the living body has a light source (first light source) 3 and a detector (first detector) 4. The outside 2 of the living body also has a light source (second light source) 5 and a detector (second detector) 6. FIG. 1 shows a plurality of light sources and detectors being used for measuring a living body. A measurement light beam (inside→outside) 7 is a measurement light beam (first measurement light beam) that is emitted from the light source 3 of the inside 1 of the living body and is detected by (enters into) the detector 6 of the outside 2 of the living body. A measurement light beam (inside→inside) 8 is a measurement light beam (second measurement light beam) that enters from the light source 3 of the inside 1 of the living body to the detector 4 of the inside 1 of the living body. A measurement light beam (outside→inside) 9 is a measurement light beam (third measurement light beam) that enters from the light source 5 of the outside 2 of the living body to the detector 4 of the inside 1 of the living body. A measurement light beam (outside→outside) 10 is a measurement light beam (fourth measurement light beam) that enters from the outside 2 of the living body to the detector 6 outside of the living body.

Next, biological measurement according to this embodiment will be described. A light source and a detector are disposed at predetermined positions outside the living body; the detector detects light emitted from the light source. The measurement light beam (inside→outside) 7 is emitted from a light source inside the living body, and advances while being absorbed and scattered by the living body, eventually the light beam 7 exits to the outside of the living body and is detected by a detector in the outside of the living body (transmissive DOT). The measurement light beam (inside→inside) 8 is also emitted from a light source inside a living body and advances inside the living body. However, this light beam 8 (which is subject to scattering and diffusion) returns toward the living body (this is called reflected light), and is then detected inside the living body (reflective DOT) by a detector 4 therein. The measurement light beam (outside→inside) 9 travels in a path inverse to the measurement light beam (inside→outside) 7 and is used for a transmissive DOT using the light source 5 outside the living body. The measurement light beam (outside→outside) 10 is reflected at the boundary B and travels in a path substantially inverse to the measurement light beam (inside→inside) 8. That is, the measurement light beam (outside→outside) 10 is used for reflective DOT using the light source 5 and a detector 6 outside the living body. According to this embodiment, the measurement light beams 7 to 10 are used and coordinated for the measurement. The data to be acquired is data such as an intensity of light in each of the detectors. Various forms of coordination are available, and those measurements may be combined variously. Basically, those measurements are performed in parallel, and details will be described according to a first example. The other forms of coordination excluding this embodiment will be described according to second and third embodiments. According to this embodiment, an organ or tissue positioned in a deep part inside a living body, such as the pancreas is to be measured. The DOT measurement may use continuous light, modulated light, or pulse light. A time domain or a frequency domain may be measured.

A plurality of light sources and detectors may be used, but single light source and detector may be used. The light source may be a laser light source with a solid-state laser, a semiconductor laser, or a gas laser, a lamp light source such as a halogen lamp, or a semiconductor light source such as an LED or an SLD (Super Luminescent Diode). At least one of these light sources may be combined with an optical fiber or the like, and light emitted from the fiber may be used as a light source. A light source inside a living body may be acquired by mounting the light source on a capsule endoscope or the like and be brought into the living body so that measurement may be performed through a body cavity such as the digestive tract. Alternatively, the optical fiber may be brought into a body for the measurement. A light source outside a living body may be implemented by applying the light fiber to the living body for measurement, or the light emitted from the light source is directly irradiated for the measure. The wavelengths of the light radiated from a light source may be equal inside and outside the living body or may be different. However, wavelengths may be different between inside and outside the living body for distinguishing them. Different parameters excluding the wavelengths, such as modulation frequencies for modulated light, pulse widths for pulse light, duty ratio, repetition frequencies, may be different between light sources inside and outside the living body.

The detector may be any detectors such as a photodiode (PD), a photomultiplier, a phototube, and a piezoelectric detector. Like the light source, the detector may be mounted on/in a capsule endoscope inside the living body for the measurement. A self-propelling mechanism, an orientation control mechanism, a detention mechanism or the like may be added to the capsule so that the capsule may be moved to a desired location for biological measurement. Alternatively, light from an optical fiber brought into the living body may be captured and guided to a detector. Because a photomultiplier light increases the size of the apparatus, it may be used as an external detector.

According to this embodiment, the control over the devices, data processing and so on are implemented by a central processing unit (e.g., microprocessor). The light sources and detectors are connected to a control unit of the central processing unit. The control unit controls operations such as light emission and detection. The control unit may further control a mechanical operation by each of the device (such as an operation inside the living body by a light source capsule) and control a series of measuring steps in association with the measurement operation. The signal acquired by the measurements is transferred to a data processing unit of the central processing unit, and the data undergoes processing and analysis there. The transferred data undergoes computer processing, and spatial distributions of the physical property values (absorption coefficient and scattering coefficient of light) of the living body are calculated. The data processing is performed by a simulation of the inverse problem, for example. The spatial distributions of the calculated absorption coefficient and scattering coefficient are imaged and are output from an output device (such as a display) associated with the processing unit.

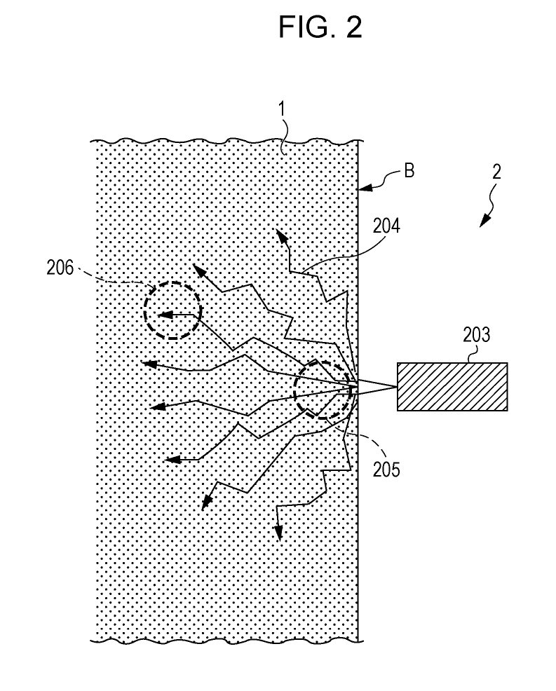

Here, a configuration according to this embodiment will be described which is adjusted to improve its resolution. As described above, light that irradiated onto a living body propagates and spreads while reducing the intensity for the absorption and scattering by the living body. Since tissues of a living body has a strong forward scattering characteristic, light spreads substantially as spherical waves in parts excluding a part extremely close to its surface. FIG. 2 schematically illustrates how the light spreads inside-the-living-body 1, when a light source 203 disposed outside-the-living-body 2 emits a measurement light beam 204. Areas 205 and 206 have an equal volume inside the living body, but these areas are located at different depths from the boundary B. More specifically, these areas are called a vicinity area 205 and a deep area 206 with reference to the distance from the boundary B or the light source 203. The measurement light beam 204 exhibits the state that light is scattered while spreading like spherical waves in a volume of interest. With conventional DOT in which light enters from the outside of a living body and a detector outside the living body detects it, the light is absorbed and scattered while it is passing through the living body. Thus, the light reaching a deep area 206 is weak. The influence of scattering and absorption is particularly high as light propagates deeper within the living body. As illustrated in FIG. 2, a large part of incident light passes through the vicinity area 205 while the light passing through the deep area 206 is a small part of the incident light. Thus, a large amount of information on the near area 205 may be acquired by collecting the scattered light passing through the near area 205 with all detectors though the resulting light is weak. However, only a small amount of information on the deep area 206 may be acquired. Therefore, according to DOT, because of the influence of the scattering, as the distance from a light source increases, the resolution of physical property information deteriorates essentially.

Therefore, according to the present invention, a light source is disposed within the living body, and light is radiated from the vicinity of a deep part inside the living body for achieving a higher resolution. In other words, the measurement of a deep part by using a light source inside a living body allows measurement of a high-resolution image of the deep part inside the living body. In this case, the present invention improves the resolution for a deep part inside a living body in coordination with DOT using a light source outside the living body on the basis of the finding that information on a part near a surface of a living body is also important as will be described below for measuring from a deep part by using a light source inside the living body.

As described above, in a DOT measurement, light propagation is described on the basis of a model of a transport equation of light inside a living body and solves the inverse problem to reconstruct a physical property value distribution of the subject. In this case, a predetermined boundary condition may be required to set in accordance with the subject. When the subject is significantly deep, it may be considered as infinite depth and an infinite boundary is applicable. However, when a human body is measured with an internal light source in the digestive tract, for example, the distance from the biological surface is about 3 to 10 cm at most, in which case an infinite boundary is not applicable. Therefore, a boundary condition may be required to set for a biological surface, information on the form and physical property values (absorption, scattering) of the boundary may be required. Acquiring this kind of information by DOT outside a living body and setting a proper boundary condition affects the accuracy of a high resolution measurement even when a light source inside a living body is used. Because a living body is in substantially constant movement due to breathing, for example, information on a surface of the living body may be required to measure continuously, and the measurement on a deep part of the living body and data acquisition at the same time may be required. Thus, measurement with a light source inside a living body in coordination with DOT using a light source outside the living body can effectively raise the accuracy of the DOT measurement using a light source inside the living body and improve the resolution of a deep part inside of the living body. Furthermore, the data from a DOT measurement outside a living body are usable simply in order to increase the number of data sets, which may further improve the resolution.

### Second Embodiment

According to a second embodiment, configuration examples of a biological measuring apparatus and a biological measuring method will be now described. According to this embodiment, a light source outside a living body is used to identify the positions of a light source and a detector inside the living body. According to the present invention, when a light source and a detector are provided inside the living body, it is difficult to directly observe their positions from the outside of the living body. Because DOT measurement may require the identification of a positional relationship between a light source and a detector, identifying the positions of a light source and a detector inside a living body is important. For example, as described above, when a light source and a detector are mounted on a capsule endoscope, for example, and the light source and detector within a body cavity are used for measurement, the identification of the position of the capsule may allow locating the positions of the light source and detector. Many technologies for identifying the position of a capsule within a body cavity have been proposed in the field of capsule endoscope. One of the proposed methods may include deducting an approximate position of a capsule from the elapsed time from when the capsule is swallowed, measuring a surrounding environment parameter (such as pH), and detecting an electromagnetic wave from an electromagnetic wave source mounted on the capsule outside a living body. However, those capsules are for identifying its approximate position, more accurate identification of the position of a capsule (more correctly, the positions of a light source and a detector mounted thereon or therein) in real time may be required for a DOT measurement. The identification of the position of the capsule is more important when the capsule has a self-propelling mechanism or a detention mechanism.

According to this embodiment, DOT measurement is implemented on light source and a detector inside a living body by using a light source outside the living body to identify their positions and thus identify the positional relationship between the light source and the detector inside and outside the living body. In this case, in order to identify the position of an apparatus inside the living body, a measurement light beam (outside→outside) 10 in FIG. 1 is used which may be measured by an apparatus outside the living body. As described above, because the resolution of DOT is lower in a deep part inside the living body, it is not enough for biological measurement. However, it is usable for identifying the positions of a light source and a detector inside a living body. This allows position detection with higher accuracy, compared with position detection based on the intensity of radio waves emitted from the inside of the living body. The identification of the positional relationship between the light sources and detectors inside and outside the living body may improve the measurement resolution according to the first embodiment and further may reduce the convergence time of data processing and reduce the time taken for a series of the measurements.

### Third Embodiment

According to a third embodiment, there will be described configuration examples of biological measuring apparatus, biological measuring method having different forms from those of the first and second embodiments. According to this embodiment, a light source outside a living body is used to identify a measurement target region in the living body when a light source inside the living body is in use. The measurement using a light source inside a living body may require grasping the structure of the living body in advance by a measurement with a different measuring device (such as MRI) in order to determine to which part and from which direction the light to radiate inside the living body. However, because pre-measurement takes a certain amount of time, the measurement target region may be identified substantially at the same time with a main measurement. In order to do so, according to this embodiment, DOT measurement using a light source outside a living body is used to identify the measurement target region with a light source inside the living body. As described above, DOT measurement on an area in the vicinity of a light source exhibits a higher resolution. However, as illustrated in FIG. 2, the measurement range is narrow in an area in the vicinity of a light source though the measurement resolution is high. Conversely, the measurement on an area remote from a light source exhibits a low resolution but a wider measurement range. Thus, DOT measure with a wide range and a low resolution using light source outside a living body is only used to roughly measure an area in a deep part inside the living body part first, and then the subject measurement range is identified. After the position and direction of a light source inside a living body are adjusted to match with the identified subject measurement range, the measurement with a high resolution using the light source inside the living body is performed. As described above, because a measurement range with a light source inside a living body is narrow, a subject measurement range may be identified in advance, and the position and/or direction may be adjusted for measurement with highly efficient radiation of light.

Also according to this embodiment, like the first and second embodiments, when a measurement is performed by using a capsule endoscope having a light source and/or a detector, a self-propelling mechanism, an orientation control mechanism may be added to the endoscope. The adjustment of the position and/or direction of the capsule by using it or them allows highly efficient radiation of light to a subject measurement range. Furthermore, a measurement with a capsule inside the living body whose position is navigated to an optimum point may be achieved by alternately performing identification of a subject measurement ranges using a light source outside a living body and the adjustment of the position and/or orientation of the capsule inside the living body.

### EXAMPLES

Examples of the present invention will be described below.

### First Example

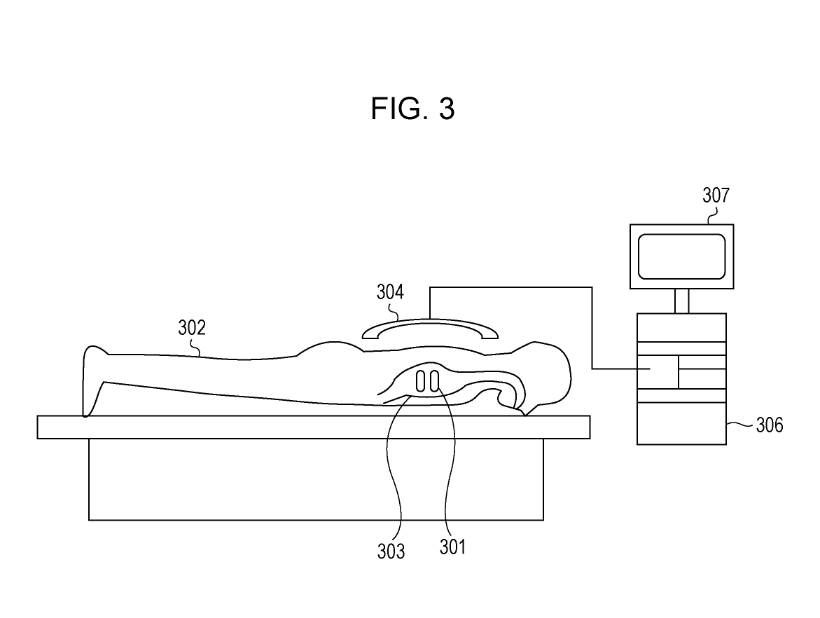

As a first example, a configuration example of a biological measuring apparatus applying the present invention will be described with reference to FIG. 3. Referring to FIG. 3, an internal measuring apparatus 301 stays within the body of a patient and has an internal light source which emits a measurement light beam within the body and a detector. The internal measuring apparatus of this example is brought from the mouth to the body of a subject 302 and operates within the digestive tract 303 of the subject 302. An external measuring apparatus 304 has an external light source that emits a measurement light beam from the outside to the inside of the body of the subject and an external detector array (not shown). In this example, the external measuring apparatus is fixed to a movable support (not illustrated) provided to a gantry (bed) on which the subject lies. A central processing unit 306 has a control unit which transmits an instruction to a devices attached to the apparatus and a data processing unit which processes and saves acquired data. A tomographic image measured, data-processed and generated by the present system is displayed on a display 307. In this example, an organ or tissue positioned in a deep part inside the body, such as the pancreas, is to be measured.

The measurement steps by the measuring apparatus will be described more specifically below. The subject 302 first takes orally (swallows) the internal measuring apparatus 301. The swallowed internal measuring apparatus reaches the inside of the digestive tract of the subject 302. Here, the internal measuring apparatus 301 moves forward within the body cavity with the movement of the digestive tract of the subject until it reaches a target position inside the digestive tract of the subject 302. When the control unit of the central processing unit 306 transmits an instruction at the time when the internal measuring apparatus 301 reaches the target region, the internal measuring apparatus 301 stays at the region instead of being carried by the movement of the digestive tract. In this example, the internal measuring apparatus stays within the stomach, and the measurement subject is the pancreas and the surrounding tissue. The instruction is transmitted by radio. Radio may be used to transmit various instructions from the control unit of the central processing unit 306 to the internal measuring apparatus. The internal measuring apparatus has a detention mechanism for staying within a body cavity without being carried by the movement of the digestive tract. When the internal measuring apparatus 301 stays within a body cavity, it starts measuring in coordination with the external measuring apparatus 304 that is provided outside the body. In this example, two measurement light beams are used, which are a measurement light beam (inside→outside) 7 and a measurement light beam (outside→inside) 9 that are illustrated in FIG. 1. The light radiated by light sources mounted on the internal measuring apparatus 301 and external measuring apparatus 304 are infrared light having wavelengths of 820 nm and 780 nm. These light sources are modulated with frequencies of 10 MHz and 50 MHz.

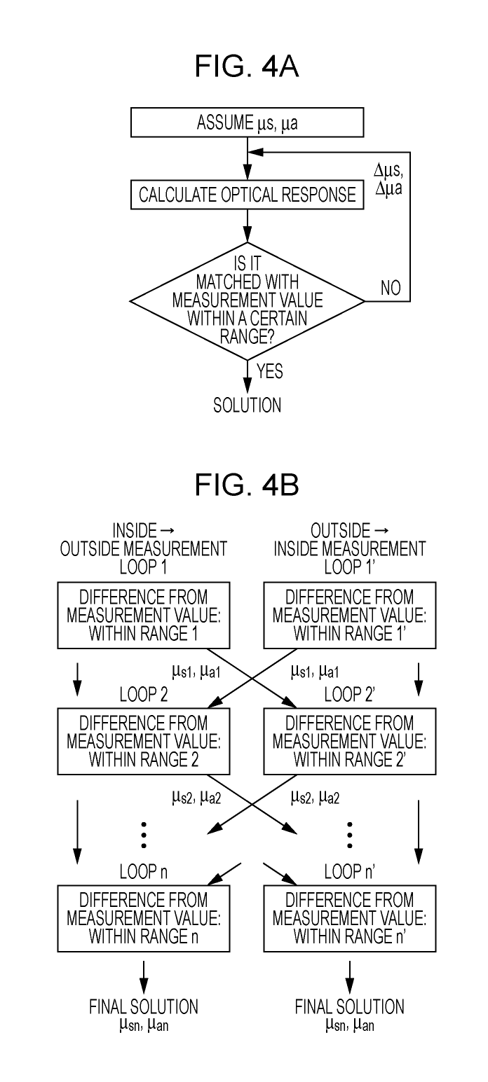

In this example, the measurements using the two light beams are performed simultaneously and output measurement data sets. In other words, a transmissive DOT measurement using light sources inside and outside the body is performed. A DOT measurement uses two values of the intensity of light and phase when modulated light is used. More specifically, the detectors measure data on time changes of the light intensity and derive the information on the phase through numerical value processing. The data sets on time changes of the light intensity acquired by the detectors inside and outside the body are transferred to the data processing unit of the central processing unit. In this example, the data transfer here is also implemented by radio. On the basis of the measurement data, the data processing unit reads data on the intensity of light and data on the phase. On the basis of these data sets, the physical property constant (scattering coefficient μₛ, absorption coefficient μₐ) distribution of the subject measurement range is reconstructed. In this example, an inverse problem method is used where the optical response acquired from the deducted distribution μₛ, μₐ is simulated, and the optimization is repeated until the response is matched with an experiment value. With reference to FIGS. 4A and 4B, reconstruction processing steps of the physical property constant distribution of this example will be described. FIG. 4A illustrates one series of physical property constant processing steps in DOT measurement. In the data processing, the optical response of the system is simulated for the deducted value μₛ, μₐ (direct problem). The physical model used here assumes and simplifies isotropic scattering of light on the basis of the transport equation of light as described above. Various simulation methods are available such as a finite element analysis and a Monte Carlo method. In this example, a finite element analysis is used. For the solution of the direct problem, if it is matched with a measured value within a certain range, the corresponding μₛ, μₐ is handled as the solution. If not, the direct problem is solved again with μₛ, μₐ that are slightly changed, and the loop is repeated in the direction that the discrepancy between simulation and measured value goes smaller, until the matching is obtained.

In order to increase the precision of the measured values, measurements may be performed again at some midpoint in the loop. In this case, for higher precision of measured values, the measurement may be performed again after the positions of the internal and external measuring apparatuses and the positional relationship between the apparatuses are manually or automatically adjusted. The S/N ratio, standard error or the like of data may be used as an index representing the precision of a measured value.

In this example, data exchange is performed between a measurement by using an internal light source and a measurement by using an external light source. How it is performed will be described with reference to FIG. 4B. Each of the boxes in FIG. 4B represents a step in FIG. 4A. First, for a measurement by using an internal light source (measurement with a measurement light beam (inside→outside) 7 in this example), a certain range is set as the differences between simulation results and measured values. This is RANGE 1 described in the step of LOOP 1 at the left column. In the same manner, for a measurement with an external light source, RANGE 1′ is set in LOOP 1′. The loops are executed such that the differences from the measured values may converge in the ranges, and the μₛ, μₐ derived from the measurements are reconstructed. As described above, because μₛ, μₐ near a boundary condition is important for highly accurate reconstruction, the μₛ, μₐ near the body surface measured with an external light source is used as an initial condition for the calculation for the next internal light source measurement to improve the calculation accuracy. In the same manner, the μₛ, μₐ appearing in the loop with an internal light source is also applied to the loop for a measured value with an external light source. By setting for the LOOPs 2 and 2′ the range of the differences with measured values as RANGE 1>2 (1′>2′), their accuracy may be increased from LOOPs 1 and (1′). The execution of these loops a plurality of number of times, the accuracy of the reconstruction may be gradually increased.

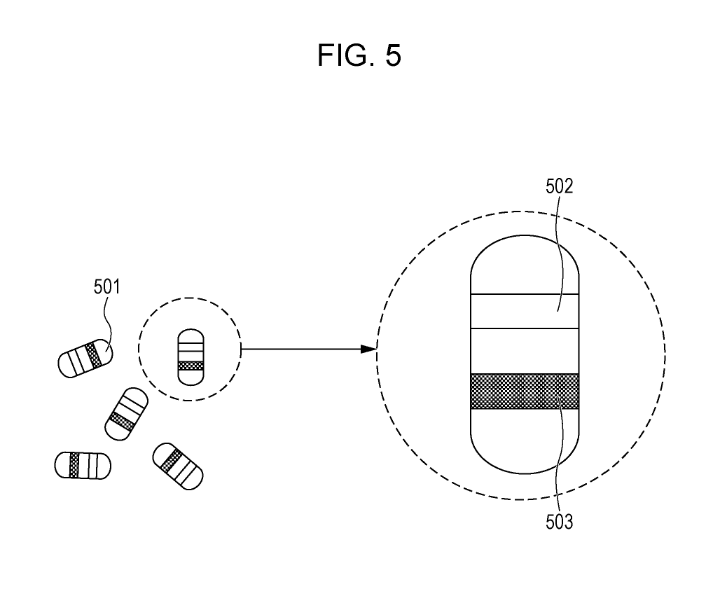

Next, an internal measuring apparatus in this example will be described with reference to FIG. 5. This example applies an internal measuring apparatus 501, a light source 502 and a detector 503. In this example, the light source and the detector are mounted on the same capsule endoscope. The light source may be an infrared region semiconductor laser, and the detector may be a PD. In this example, both of the light source and the detector are regularly disposed on the circumference of the capsule so that they can handle light beams from all directions. In this example, the number of capsules is 10, and the light sources and detectors thus function with 10 channels for measurement with a predetermined arrangement. The positions and arrangements of the capsules may be arbitrarily changeable by adding a self-propelling mechanism to the capsules. Performing the measurement again after changing the locations of the capsules allows measurements at more points. Alternatively, after a reconstructed image as a result of one measurement is output, the position and/or angle of the internal measuring apparatus may be changed for measurement under an ideal condition. No upper limit is provided for the numbers of light sources and detectors for the measurement. However, an excessive number of light sources and detectors may prevent easy swallowing for use. At least one light source and one detector are enough. In that case, the capsule may be moved many times to acquire many data sets, which may increase the measurement time. However, a subject may swallow it easily, compared with swallowing a plurality of capsules. Functions (such as photographing, tissue sampling, injection), other than a light source and a detector, of a general capsule endoscope may be added. Power may be supplied externally by radio waves when those functions may be mounted in a capsule body as in a general capsule endoscope.

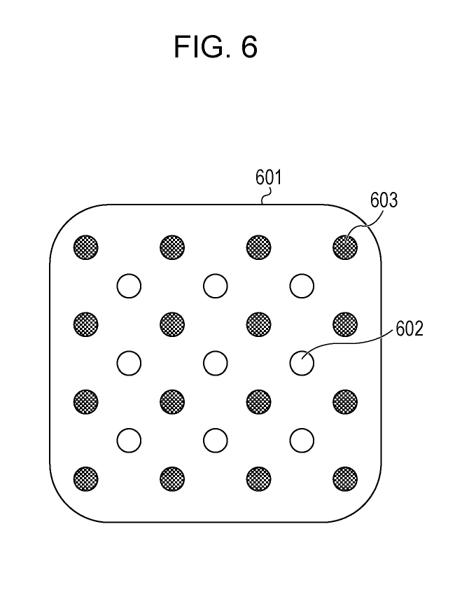

Next, an external measuring apparatus according to this example will be described with reference to FIG. 6. There are provided an external measuring apparatus 601, light sources 602 and detectors 603. In this example, the external measuring apparatus has a panel shape and has light sources and detectors on its surface. The light sources and detectors are disposed in an array, and a light source is disposed between detectors. Each of the intervals between the light sources and between detectors is 2.5 cm. 9 light sources and 16 detectors are provided. However, this is given for illustration purpose only. Like the internal measuring apparatus, no limit is provided for the number of light sources and detectors. In this example, the light sources and detectors of the external measuring apparatus may be semiconductor lasers and PDs like the internal measuring apparatus. According to this example, the light sources and detectors are integrated into a panel shape, but they may be separated. As described above, the panel is fixed to a bed on which a subject lies. However, by moving it instead of fixing, measurements may be performed at different locations, like the internal measuring apparatus. Furthermore, in this example, the light beams to be used are only the measurement light beam (inside→outside) 7 and measurement light beam (outside→inside) 9 as illustrated in FIG. 1. But one or two of the remaining measurement light beam (inside→inside) 8 and measurement light beam (outside→outside) 10 may be added. The data processing method in that case will be described with reference to the second example.

In this example, the measurement light beams have different wavelengths inside and outside the body. According to this example, the detectors inside and outside the body may be used for measuring light emitted from light sources inside and outside the body, respectively. But they may detect light beams emitted from the internal and external light sources respectively as stray light. In order to distinguish the stray light, the light beams emitted from the internal and external light sources may have different wavelengths. In this case, a light wavelength filter, for example, may be used for distinction between them. The modulation cycles may also differ inside and outside the body from the viewpoint of the distinction between light beams from the internal and external light sources. In this case, the distinction between light beams from the internal and external light sources may be allowed not only by differentiating the wavelengths but also by demultiplexing measured data from frequency component in measured data processing or using an electric frequency filter, for example.

According to this example, both of the internal and external light sources emit modulated light in predetermined cycles, but one or both of them may be continuous light. They may emit pulse light. In an expanded sense, continuous light is included within modulated light as the light with infinite modulation period. The amount of data decreases in order of continuous light, modulated light, and pulse light, the time for acquiring data may be saved in the order. A highly sensitive photomultiplier may be more easily used as the external detector, the internal light may has short pulses in the order of picoseconds for time-resolved measurement, which increases the number of data sets and may improve the resolution. Because it is difficult to use a high-sensitive detector as an internal detector, the use of continuous light or modulated light is convenient. The data in the vicinity of the boundary may be acquired by a measurement using an external light source at the same time as a measurement using an internal light source, like this example, and the both of the data sets may be exchanged. This may increase the accuracy of the measurement using an internal light source and increase the resolution. Particularly, it is important to execute a measurement using an external light source and a measurement using an internal light source in parallel. This may always provide data sets measured inside and outside the body at the same time even with movements by the digestive tract and/or breathing.

### Second Example

A configuration example that is different from the first example will be described as a second example. In this example, all of the four light beams illustrated in FIG. 1 are used for measurement. In other words, a reflective DOT is performed in addition to a transmissive DOT using internal and external light sources as performed in the first example. FIG. 3 illustrates an entire diagram of the measuring apparatus like the first example. The difference from the first example in data processing after a measurement will be mainly described. In this example, the measurements using internal and external detectors may require detection of signals of both transmissive DOT and reflective DOT. Thus, as in the first example, the signals may have different characteristics in order to distinguish the internal and external light sources. More specifically, signals may have different wavelengths since they may be distinguished by using a light wavelength filter. The signals may have different modulation frequencies, which allow their demultiplexing in data processing. Also in this example, the light sources to be used emit infrared modulated light, which is the same as the first example.

However, regarding detectors, a different detector is used for each light source (for each wavelength) inside and outside the body in this example. Therefore, two types of detectors are provided inside and outside the body, and signal light beams of a reflective DOT and a transmissive DOT using the light sources inside and outside the body are separately monitored. The data to be measured by the detectors is changes of the light intensity to time as in the first example, and the data is transmitted from the detectors to the data processing unit of the central processing unit. The data processing unit reads measured data from each of the detectors and reconstructs a distribution of a scattering coefficient μₛ, absorption coefficient μₐ.

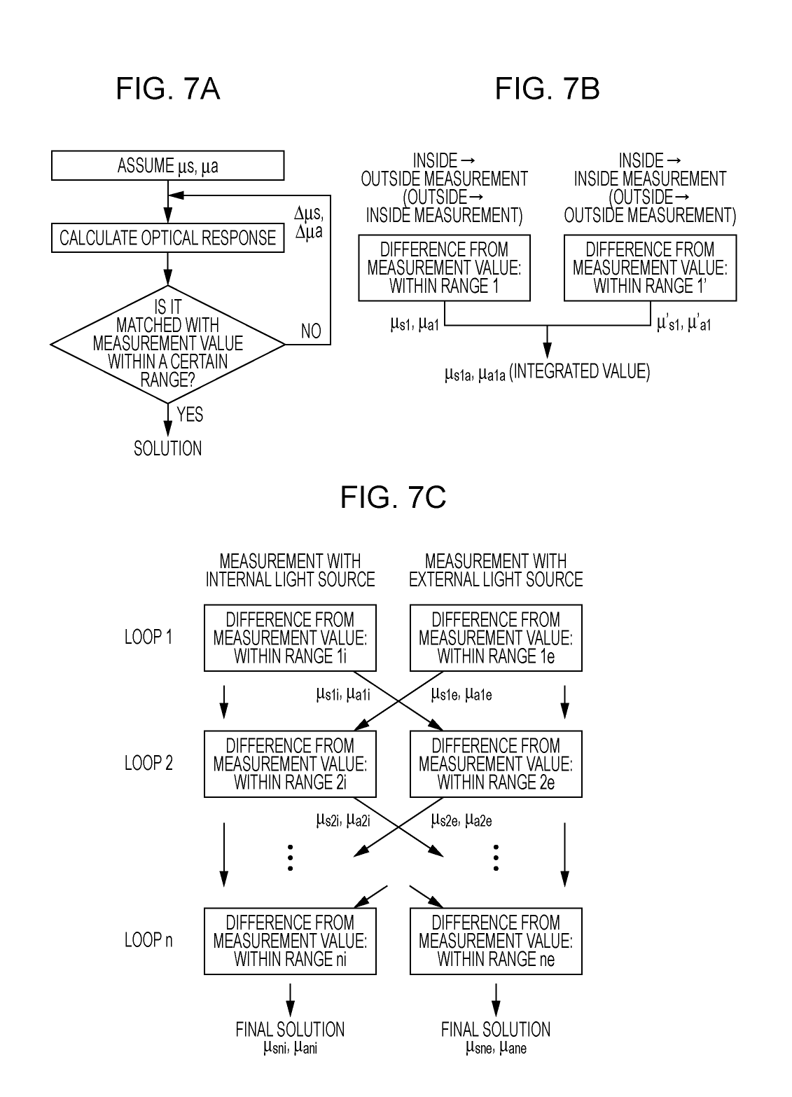

In this example, because four types of measured data are used, the reconstruction processing is as in FIGS. 7A to 7C. The reconstruction processing is first performed on the four measured data sets corresponding to four types of measurement as in FIG. 7A. This is a step of searching a physical property value at which a simulated value and a measured value are matched in a certain range, which is exactly the same as FIG. 4A of the first example to every type of measurement. Next, in this example, data processing is performed on measure values wing an internal or external light source. This is a step in FIG. 7B. Referring to FIG. 7B, physical property values derived from the data sets of transmissive and reflective DOT measurements using internal (external) light source are merged and are output as one physical property value. The illustrated boxes in FIG. 7B represent steps in FIG. 7A. The transmissive and reflective DOTs output the same physical property value if the same light source is used. However, because the main paths of the measurement light beams are different, their reliabilities differ. Therefore the merging steps are necessary and the way of them are as described below.

Fundamentally, transmissive DOT allows light reaching a deep part more easily than reflective DOT. However, the measurement range may easily be narrowed in the lateral direction in a shallow part. On the other hand, reflective DOT may not allow light reaching a deep part easily, but the measurement range may easily be extended in the lateral direction in a shallow part. Therefore transmissive DOT is suitable for measuring an image of a deep part in a narrow range, and reflective DOT is suitable for measuring an image in a shallow area in a wide range. Thus, the processing of merging physical property values derived from them may be implemented by calculating specific weighted average based on their reliability instead of averaging between them. Next, physical property values derived from the internal and external measured values are used in steps in FIG. 7C so that the accuracy of the entire physical property values may be increased. The processing to be performed is the same as the step in FIG. 4B of the first example to each of physical property values derived by the measurement with internal and external measured value in FIG. 7B, and the processing within one box corresponds to a step in FIG. 7B.

Furthermore, in this example, another effect is provided by the use of a reflective DOT using an external light source. In this example, reflective DOT using an external light source, that is, the measurement light beam (outside→outside) 10 illustrated in FIG. 1 is applied to measure the position of a capsule inside a body. As described above, positional information may thus be detected. Because the positional information is recorded at the same time when it is measured, it is used for the reconstruction based on the corresponding measured data, which may increase the accuracy of the reconstruction. Particularly, when a self-propelling mechanism is added to the internal measuring apparatus and the measuring apparatuses inside and outside the body are moved for measurement at the same time, positional information in synchronization with the measurement may be acquired. Thus, the positional information identification function with DOT is important. The detection of the positional information on the capsule allows external monitoring of that the capsule has reached a target position inside the body. Thus, the arrival of the capsule may be more securely determined rather than the deduction of the position of the capsule based on the elapsed time after it is swallowed as in the first example.

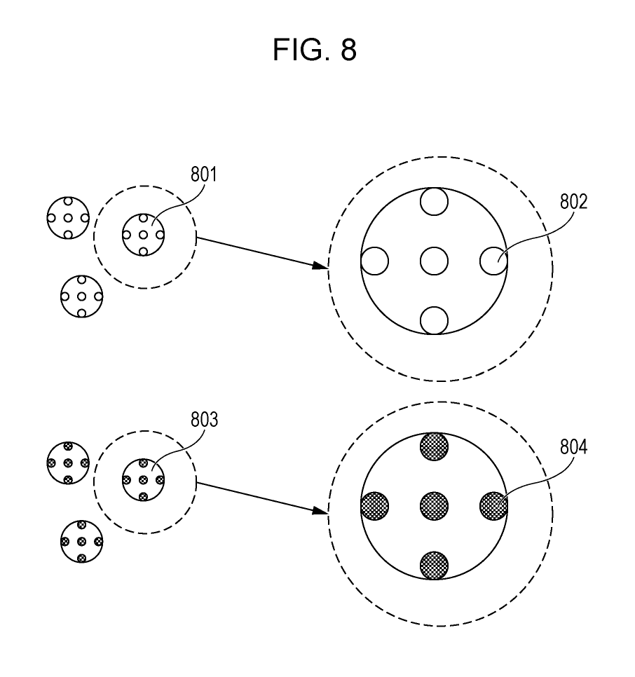

Next, the internal measuring apparatus according to this example will be described. FIG. 8 is a schematic diagram of the internal measuring apparatus according to this example. There are provided a capsule 801 for a light source of the internal measuring apparatus, a light emitting surface 802, a capsule 803 for a detector in an internal detecting device, and a detector surface 804. In this example, the internal measuring apparatus has functions for a light source capsule and a detector capsule. The light source capsule and detector capsule have different functions from each other. The light source capsule is globular as illustrated, and light source surfaces are provided at six positions in ±x, y, z directions so that light may be radiated isotropically. The detector capsule is also globular and has detector surfaces at the same positions as the light source surfaces of the light source capsule. The light sources and detectors are semiconductor lasers and PDs, as in the first example. In this example, as described above, because detectors for an internal light source and for an external light source are provided, there are two types of the detector capsules corresponding to two types of wavelength. In this case, the wavelength of each of the detector may be selected through a filter (not illustrated) covering the detector surface of the detector. Regarding the number of capsules, 10 light source capsule and 10 detector capsules for each of the internal light sources and the external light sources are provided. In this way, the detector capsules may detect light beams having different wavelengths and/or characteristics so that the measurement light beams from the light source inside and outside the body may be processed separately and simultaneously. Like the first example, a self-propelling mechanism for a capsule and/or other functions of a general capsule endoscope may be added.

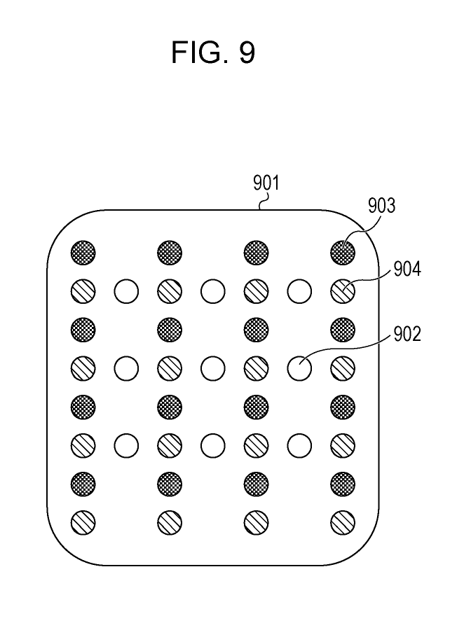

Next, an external detecting device according to this example will be described with reference to FIG. 9. There are provided an external measuring apparatus 901, a light source 902, a detector 903, and a detector 904. The detectors of the external measuring apparatus are also specific to the corresponding light sources inside and outside the body, like the internal measuring apparatus. The detector 903 is for an external light source, and the detector 904 is for an internal light source. In each of the detectors, a wavelength is selected through a light wavelength filter, not illustrated. The intervals between the light sources and detectors are 2.5 cm, like the first example. As described above, in this example, all of the four types of light illustrated in FIG. 1 are used. All information pieces acquired from them are used to reconstructs the physical property value. Thus, the most accurate reconstructed image may be output.

### Third Example

A configuration example that is different from the aforementioned examples will be described as a third example. According to this example, a subject measurement range is identified by a measurement using an external light source before a measurement. The fundamental apparatus configuration is substantially the same as the first (and second) examples. Only differences will be described below. In this example, the measurement light beams to be used are, like the first example, a measurement light beam (inside→outside) 7 and a measurement light beam (outside→inside) 9 illustrated in FIG. 1, and transmissive DOT with an internal light source and an external light source is applied. The configurations of the internal measuring apparatus and external measuring apparatus are the same as those of the first example.

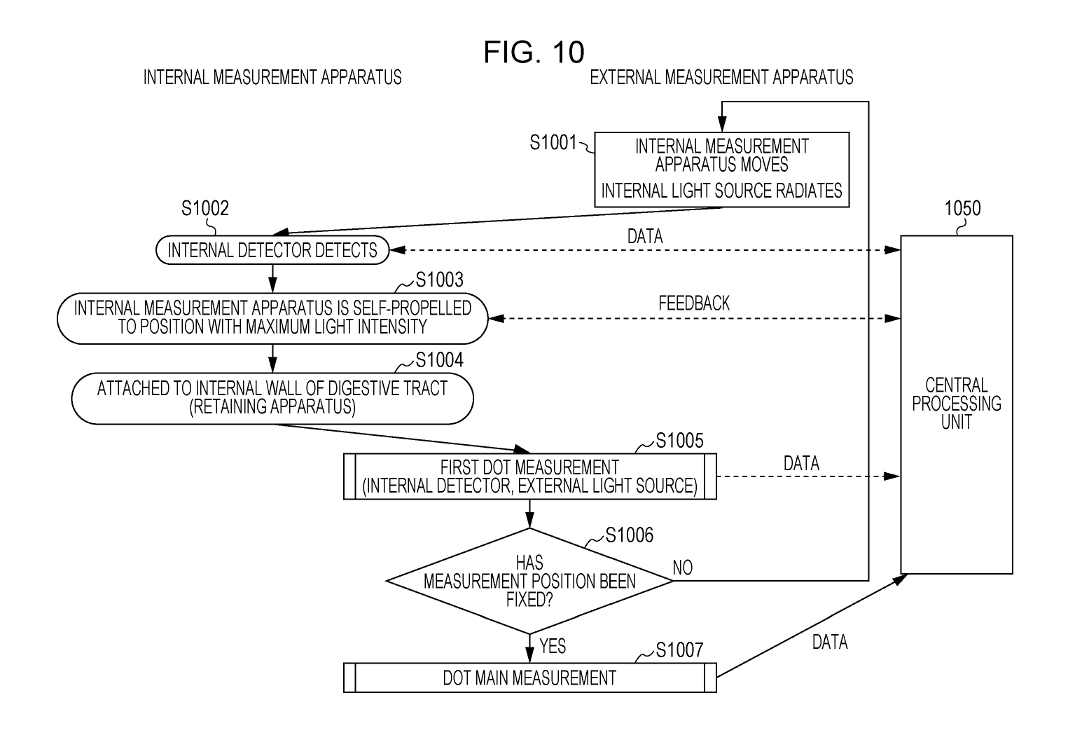

Measuring steps of this example will be described below. The data processing steps of this example are the same as those of the first example, but the measuring steps before it has a characteristic point. A series of measuring steps are illustrated in FIG. 10. In this example, the path after the capsule is swallowed until the capsule reaches the target region (inside the stomach) is the same as the first example. Here, in this example, a measurement light beam (external measurement light beam) from the external light source is first radiated to the stomach of the subject (S1001). The each capsule detects the external measurement light beam with the attached detector (S1002), and is self-propelled toward the external measurement light beam with the attached self-propelling mechanism (S1003). For example, the capsule having the internal measuring apparatus is self-propelled toward the internal wall of the digestive tract, and remains attached thereto by means of a retaining apparatus (S1004). The self propelling is implemented in the direction that the quality of light detected by the detector increases by using feedbacks between the detected intensity of light and a self-propelling instruction. The detection signal processing, feedback processing and self-propelling instruction are all implemented by the control unit of the central processing unit 1050. When the capsule moves to the position with a maximum (or the highest) detected intensity of light, a first DOT measurement is performed with the external measurement light beam (S1005).

In this example, because the capsule positions inside the stomach, the intensity of light detected on the wall of the stomach in the direction of radiation from the outside of the body is at a maximum intensity. Thus, when the capsule reaches the part with the maximum intensity of light on the internal wall of the stomach, it is detected by a pressure sensor attached to the capsule. Then, the capsule is adhered to a detention mechanism attached thereto. When the capsule stops and if the measurement light beam (outside→inside) 9 is detected by the detector of the internal measuring apparatus, a measurement starts. The intensity of light data measured by the internal measuring apparatus is transmitted to the data processing unit of the central processing unit by a radio wave signal. The physical property value data-processed there is reconstructed. Because the subject region is in a deep part inside the body, a detailed reconstruction image may be not acquired from this first measurement. However, rough information on the region to be measured may be acquired. In this example, the external measuring apparatus is iteratively moved to find the region to be measured (S1006: NO). Thus, the information is used during the measuring steps, which is a characteristic point of this example. The external measuring apparatus may be moved or its angle may be changed in this example while the external measuring apparatus is fixed in the first example. Here, following the movement of the external measuring apparatus, the capsule inside the body moves by the feedback processing. After the series of steps are repeated and the position of the region to be measured is roughly identified (S1006: YES), detail measurements using internal light sources are performed next (S1007). Though, in the first example, the measurements using measurement light beams inside and outside the body are performed simultaneously, they are not performed simultaneously in this example but are performed alternately. The transfers of measurement data are also performed alternately. When data sets are acquired from the measurements, a physical property value distribution is reconstructed. The reconstruction is performed in the same manner as the steps in FIGS. 4A and 4B according to the first example.

In this example, because a measurement using internal light and a measurement using external light are performed alternately, the light source and detector of the internal measuring apparatus provided individually in the same capsule according to the first example may be configured by a single element. In this case, the internal light source functions as a detector when a detail measurement is performed with an external light source and functions as a light source when a measurement is performed with an internal light source. For that, the wavelength of the external light source may be required to be shorter than the wavelength of the internal light source. Furthermore, in this example, because of the alternate measurements with internal and external light, the measurement time may be reduced practically by using continuous light as external light and pulse light as internal light for higher resolution measurement in order to identify the subject measurement range.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-245106, filed Nov. 9, 2011, which is hereby incorporated by reference herein in its entirety.

What is claimed is:

1. An apparatus which measures physical property information on a part of a living body by diffuse optical tomography, the apparatus comprising:
at least one first light source and one first detector both disposed inside the living body;
at least one second light source and one second detector both disposed outside the living body; and
a processor which processes measured values and reconstructs a distribution of scattering coefficients or absorption coefficients of light in the living body,
wherein the measured values are acquired from
at least one measurement light beam emitted from the first light source and detected by one of the first and second detectors, and
at least one measurement light beam emitted from the second light source and detected by one of the first and second detectors.

2. The apparatus according to claim 1, wherein the processor processes measured values acquired from all measurement light beams emitted from the first light source and detected by the first and second detectors and a measurement light beam emitted from the second light source and detected by the first and second detector.

3. The apparatus according to claim 1, wherein a wavelength of the measurement light beam emitted from the first light source is different from a wavelength of the measurement light beam emitted from the second light source.

4. The apparatus according to claim 1, wherein a measurement light beam emitted from the first light source and a measurement light beam emitted from the second light source are modulated light and have different modulation cycles.

5. The apparatus according to claim 1, wherein:
a measurement light beam emitted from the first light source is pulsed light; and
a measurement light beam emitted from the second light source is modulated light or continuous light.

6. The apparatus according to claim 1, wherein each of the first detector and the second detector includes a plurality of detectors, each of which detects light beams having different characteristics from each other.

7. The apparatus according to claim 1, wherein the first light source and first detector include the same elements.

8. The apparatus according to claim 1, wherein the wavelength of the measurement light beam emitted from the first light source is shorter than the wavelength of the measurement light beam emitted from the second light source.

9. A method of measuring physical property information on a part of a living body by diffuse optical tomography, the method comprising:
processing measured values acquired from
at least one measurement light beam of at least one of a first measurement light beam emitted from a light source inside the living body, advances within the living body, and is detected by a detector outside the living body and a second measurement light beam detected by a detector inside the living body, and
at least one measurement light beam of at least one of a third measurement light beam emitted from a light source outside the living body, advances within the living body, and is detected by a detector inside the living body and a fourth measurement light beam detected by a detector outside the living body and reconstructs the distribution of scattering coefficients or absorption coefficients of light in the living body.

10. The method according to claim 9, wherein all measurement light beams from the first to fourth measurement light beams are processed, and a distribution of scattering coefficients or absorption coefficients of the living body is reconstructed.

11. The method according to claim 9, wherein a light source outside the living body is used to locate the position of a light source and detector inside the living body.

12. The method according to claim 9, wherein a light source outside the living body is used to locate a subject area inside the living body when the light source inside the living body is in use.
